(19) United States
(12) Patent Application Publication
(10) Pub. No.: US 2011/0217770 A1
(43) Pub. Date: Sep. 8, 2011

(54) Cell Piercing Device and Method of Using Same

(76) Inventor: Eran Bram, Ein Sarid (IL)

(21) Appl. No.: 13/128,949

(22) PCT Filed: Nov. 12, 2009
(86) PCT No.: PCT/IB09/55026

§ 371 Date: May 12, 2011

Related U.S. Application Data
(60) Provisional application No. 61/113,788, filed on Nov. 12, 2008.

(51) Int. Cl. C12N 5/00; C12M 1/00

(52) U.S. Cl. 435/325; 435/283.1

(57) ABSTRACT

A cell piercing device comprising a container, a respectively matching cap and a cell interacting surface is disclosed. The cell interacting surface comprises a plurality of extensions or prongs. The cell piercing device may further comprise a pedestal. Furthermore, a method of facilitating an interaction with the interior of cells is disclosed. The method includes the steps of loading into the container of the cell piercing device, a liquid solution containing the cells and tightening the respectively matching cap. Then centrifuging the device while the extensions or prongs protruding from the cell interacting surface are oriented essentially within the direction of the vector of the centripetal force and thereafter centrifuging the device while said extensions or prongs are oriented essentially within the direction of the vector of the centrifugal force.

### CROSS-REFERENCE TO RELATED APPLICATIONS

The present application claims the benefit of priority to U.S. Provisional Patent Application Ser. No. 61/113,788, filed Nov. 12, 2008, entitled ‘Sterile bi-directional centrifugation of living cells and bio-molecules.’ The aforementioned application is incorporated herein by reference.

### TECHNICAL FIELD

The present invention generally relates to device and method for piercing biological cells. In particular, the invention relates to device and method to be preferably implemented in molecular biology, to deliver substances into biological cells while essentially preserving structural integrity of the cells.

### BACKGROUND ART

Various techniques and methods of delivering substances into biological cells are known in the art. Some of these techniques are implemented in molecular biology and life sciences research, whereas others in medicine and cellular therapy. The common to all methods is the objective of delivering substances into biological cells while essentially preserving structural integrity and hence the vitality of the cells. Numerous of the aforementioned techniques and methods are aimed at delivering nucleic acids into the cells, inter alia including transformation, transduction, transfection, liposomes, biolistics, electroporation, etc.

WO1988/04322 discloses a method and apparatus for introducing molecules and particles inside living cells. The invention utilizes artificial gravity created by a spinning rotor to produce a single compressed cell layer against the outer flat surface; and then, by an electric pulse an opening is made on the top of the cell's membranes. The subsequent acceleration of the rotor allows the controlled extraction of the cytoplasm from inside the cells; and the deceleration of the rotor, allows the cells to aspirate through the open membrane hole the surrounding liquid containing molecules to be injected into the cells.

US2006/275371 teaches methods and materials for delivering biologically active molecules to cells in vitro or in vivo by using carbon nanotubes functionalized with a linking group that is covalently bound to the nanotubes, such as a protein. The biologically active molecule is released from the nanotube when the complex has been taken up in an endosome.

WO2008/133755 is directed to a method and device for the delivery of molecules into individual cells. The device comprises a microscopic tip attached to a mechanical scanning device for positioning the tip relative to the target cell and for moving the tip into the target cell; a nanostructure, such as a carbon nanotube, fixed on an end of the microscopic tip; and a biological molecule attached to the nanotube by a chemical linkage which is cleaved in an intracellular environment. The biological molecule may be one or more of proteins, nucleic acids, small molecule drugs, and optical labels.

It is noted however that the cell injecting device disclosed in WO2008/133755 requires a manipulator with nanoscale resolution for inserting and removing the needle. The tip is lowered into the cell so that biological molecule is inserted into the cell. After the biological molecule is released in the target cell, the tip and nanotube are removed.

Moreover, U.S. Pat. No. 7,195,780 and application Ser. No. 2004/0186459 are believed to represent the current state-of-the-art. Therefore a simple closed device for cell transfection under the biological constrains of sterility and sustention in an aqueous solution environment, to enable the recovery of intact and viable cells shall have an undisputable benefit in various clinical and research applications.

### SUMMARY OF THE INVENTION

There is provided in accordance with some embodiments of the present invention a cell piercing device comprising a container, a respectively matching cap and a cell interacting surface (CIS), disposed in the container. The CIS comprises a plurality of extensions or prongs. The cell piercing device may further comprise a CIS pedestal.

There is provided a method of facilitating an interaction with the interior of cells, the method includes the steps of: loading into the container of the cell piercing device, through an anterior opening thereof, a liquid solution containing the cells, tightening the respectively matching cap, thus sealing the container; centrifuging the device while the extensions or prongs protruding from the CIS are oriented essentially within the direction of the vector of the centripetal force and thereafter centrifuging the device while said extensions or prongs are oriented essentially within the direction of the vector of the centrifugal force.

Cells, as referred herein, should be understood as encompassing any type of biological or artificial material, particularly one forming a plurality of individual unit-like structures; examples of such biological or artificial materials inter alia include eukaryotic cells, prokaryotic cells, bacteria, spores, protozoa, nucleus, various cellular organelles, endosomes, virons, capsids, liposomes, any type of suspensions and particularly oily-aqueous emulsions, any type of micro- or nano-particles, etc.

It should be understood, however, that the description herein of specific embodiments is not intended to limit the invention to the particular forms disclosed, but on the contrary, the intention is to cover all modifications, equivalents, and alternatives falling within the scope of the invention.

### BRIEF DESCRIPTION OF THE DRAWINGS

The present invention will be understood and appreciated more fully from the following detailed description taken in conjunction with the appended drawings in which:

FIG. 1 is an exploded isometric view of an embodiment of the device of the present invention;

FIG. 2 is a cross-sectional view of an embodiment of the device of the present invention shown in FIG. 1;

FIG. 3 is an exploded isometric view of a preferred embodiment of the device of the present invention;

FIG. 4 is a cross-sectional view of a preferred embodiment of the device of the present invention shown in FIG. 3;

FIG. 5 is an enlarged isometric view of preferred embodiment of a pedestal and cell interacting surface of the device of the present invention.

While the invention is susceptible to various modifications and alternative forms, specific embodiments thereof have been shown by way of example in the drawings. The components in the drawings are not necessarily to scale, emphasis instead being placed upon clearly illustrating the principles of the present invention.

### DISCLOSURE OF THE INVENTION

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with technology- or business-related constraints, which may vary from one implementation to another. Moreover, it will be appreciated that the effort of such a development might be complex and time-consuming, but would nevertheless be a routine undertaking for those of ordinary skill in the art having the benefit of this disclosure.

### THE CELL PIERCING DEVICE OF THE INVENTION

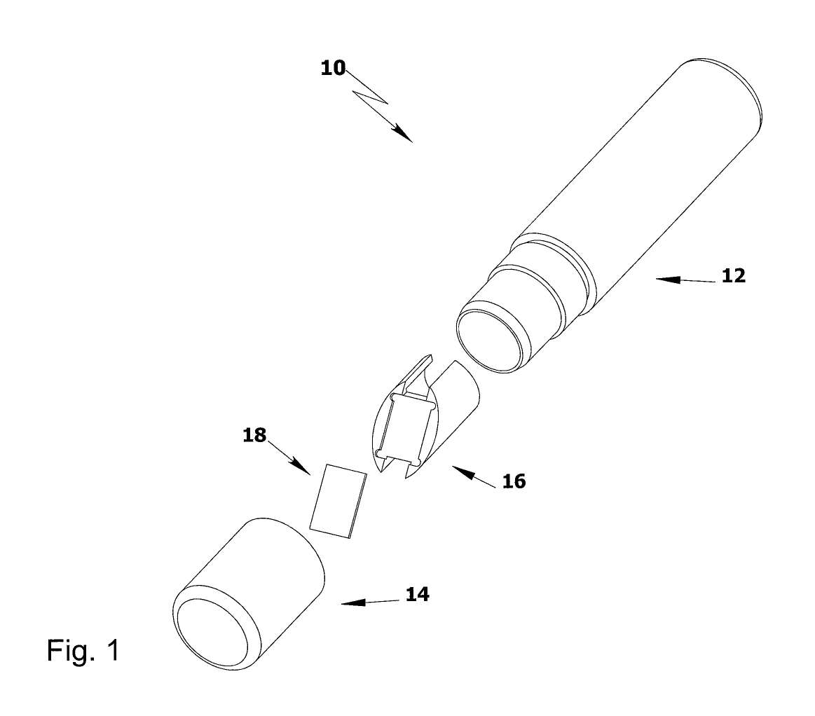

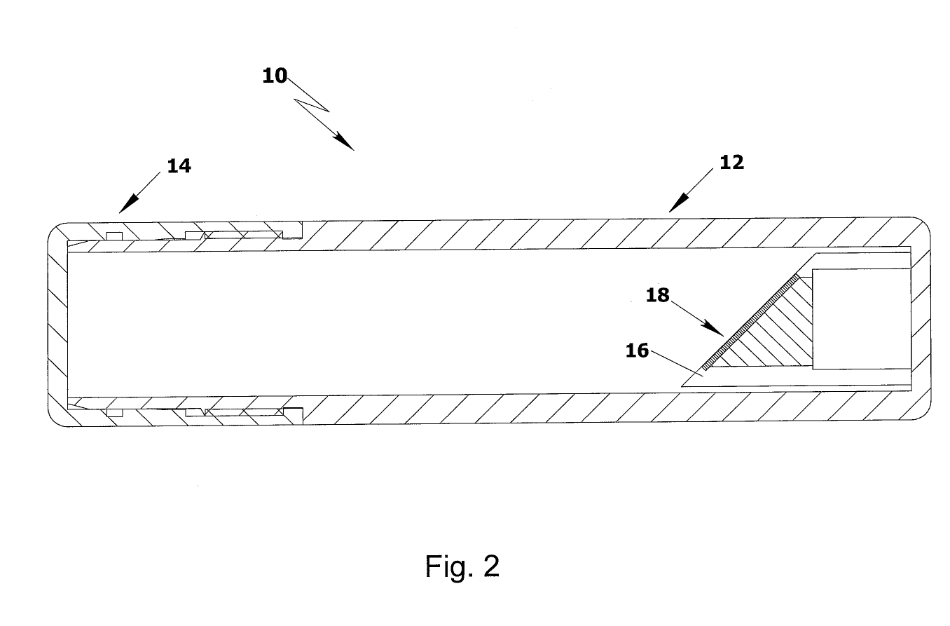

In accordance with some embodiments of the present invention, cell piercing device 10 (hereinafter CPD), shown in FIGS. 1 and 2, comprises container 12, respectively matching cap 14, cell interacting surface (hereinafter CIS) pedestal 16 and CIS 18. Container 12 is essentially cylindrical and comprises an anterior opening allowing loading and discharging of solutions. Container 12 may include one or more trunnions (not shown), adapted for accommodation of CPD 10 in standard centrifuges. Various accommodation arrangements can be employed for instance for swinging bucket centrifuge or fixed angle centrifuges. Respectively matching cap 14 is typically capable of hermetically sealing container 12, thereby preventing spillage of the liquid content thereof and penetration of contaminants and microorganisms thereto. For instance, a screw threading may be employed at the interface between container 12 and cap 14, for securing the latter to the former; it will be understood however that any conventional means known in the art, such as detachable joining, may be employed for securing cap 14 on container 12.

Slanted CIS pedestal 16 is inserted into container 12 and typically frictionally engaged vis-à-vis the interior cylindrical face thereof. CIS pedestal 16 is employed to achieve a predetermined positioning of CIS. It should be acknowledged that the slanted configuration of pedestal 16 is merely an example and thus CIS pedestal may be embodied in various other configurations or forms, in part exemplified hereunder. It should be further acknowledged that pedestal 16 or any other CIS pedestal in accordance with the present invention may be formed as an integral or monolithic portion of container 12; for example as a circumferential rim adapted to support and affix CIS 18 in an appropriate position.

CIS 18 in mounted on pedestal 16 and disposed within container 12 somewhat spaced from the bottom. CIS comprises a surface having a plurality of micro- and/or nano-scale extensions or prongs protruding therefrom, typically oriented somewhat towards cap 14. It should be acknowledged that orientation of extensions or prongs may vary and thus in some embodiments a slanted configuration of extensions or prongs may be employed, for instance depending on swinging bucket centrifuge versus fixed angle centrifuge accommodation arrangement. CIS 18 may be formed from silicon, macro-carbon molecules, metal alloys, nanotubes, nanofibers or any other composition known in the art, allowing the formation of a plurality of micro- and/or nano-scale extensions or prongs protruding from a base-plate structure and preferably arranged in a grid-like pattern. An example of a method of producing such micro- and/or nano-scale extensions or prongs protruding from a base-plate structure is disclosed by V. I. Merkulov, in ‘Patterned growth of individual and multiple vertically aligned carbon nanofibers’ as published in Applied Physics Letters, volume 76, number 24, on 12 Jun. 2000.

The extensions or prongs are preferably sized and spaced apart across the surface of the base-plate structure, accordingly to the dimensions of the cells to be pierced and depending upon the application for which the cell piercing device is employed; thus different kinds of cells and applications of the device may require extensions or prongs of different size and/or spacing across the surface of the base-plate structure. The extensions or prongs act as a physical interaction means by piercing the cells and thereby providing for an interaction between the outer surface of the extensions or prongs and the interior of the cells.

An interaction, as referred herein, is preferably of a chemical- or biochemical-character. Nevertheless, it is stressed that various other interactions are available within the scope of the device and method of the present invention; the instances of such interaction in a non-limiting manner include electrical, thermal, optical, mechanical and vibro-acoustic interactions.

It is further emphasized that the interaction between the outer surface of the extensions or prongs and the interior of the cells can be unidirectional or bidirectional. Thus a substance or compound can be released from the outer surface of the extensions or prongs into the interior of the cell or exposed thereto, thereafter referred to as delivery applications; whereas alternatively or additionally a substance or compound from the interior of the cells can be bound to the extensions or prongs or absorbed therein, thereafter referred to as collection applications.

Delivery applications are typically employed for molecular biology and cell therapy applications, in a non-limiting manner include transformation, transduction and transfection of living cells. Delivery applications typically employ molecular bio/chemical release mechanism, in which the substance to be delivered is covalently or otherwise linked to the extensions or prongs by means of a linker that is modified upon the interaction with cells' interior, so as to release the substance linked thereby into the interior of the cells. Examples of such molecular bio/chemical release mechanisms are known in the art and include oxidation-reduction mechanisms, pH-mediated mechanisms, enzymatically cleaved mechanisms, etc.

In summary, interaction as defined herein refers to any type of interaction occurring essentially between the portions of the extensions or prongs that have penetrated inside the cells and the interior of the cells.

### THE METHOD OF THE INVENTION

In accordance with some embodiments of the method of the present invention, the container of the cell piercing device (CPD) is initially loaded, via the anterior opening thereof, with solution containing cells intended to undergo an interaction, as specified hereinabove.

The respectively matching cap is then tightened on the container to seal it. The CPD is subsequently centrifuged, while the extensions or prongs protruding from the cell interacting surface (CIS) are oriented essentially within the direction of the vector of the centripetal force; thereby the cells are urged by the centrifugal force towards CIS and consequently pierced by the extensions or prongs and spitted onto them, ergo providing for the aforementioned interaction to take place. If the interaction spontaneously occurs upon the contact of the extensions or prongs with the interior of the cells, the time needed for the interaction to complete to a desired extent is allowed to lapse; whereas if the interaction does not spontaneously occur merely upon the aforementioned contact, the action needed to induce or initiate the interaction is performed.

Upon the completion of the interaction, the CPD is centrifuged, while the extensions or prongs protruding from the cell interacting surface (CIS) are oriented essentially in the same direction of the vector of the centrifugal force; thereby the cells are urged by the centrifugal force away from the CIS and consequently driven off the extensions or prongs and released into the interior of the container. Thereafter, the cap is removed from the container and the cells underwent the interactions can be collected.

### BEST MODE FOR PRACTICING AND CARRYING OUT THE INVENTION

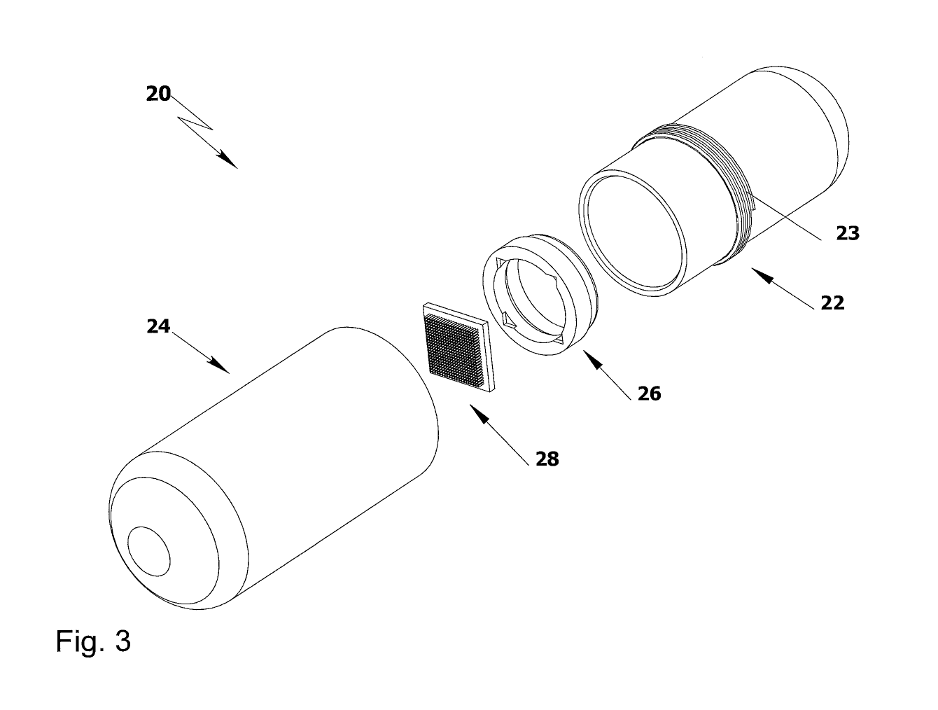

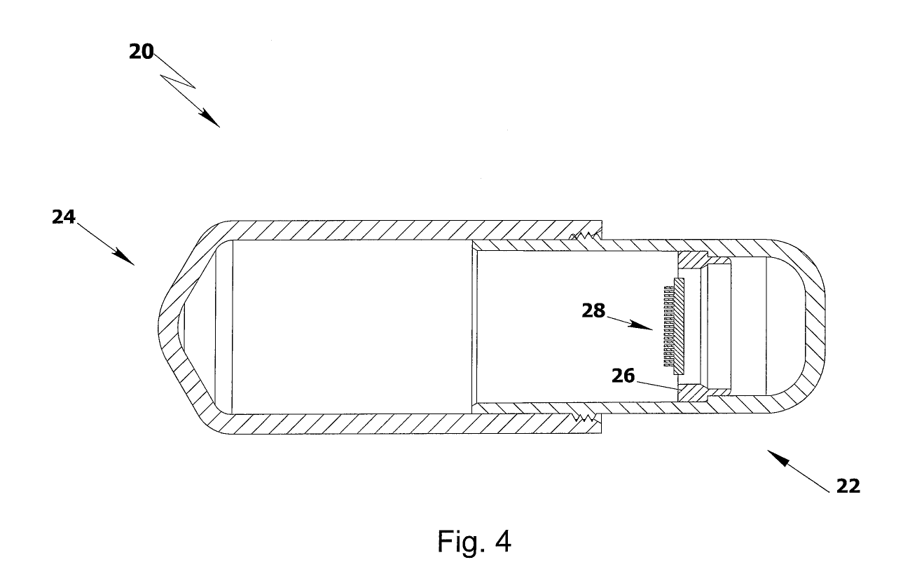

In accordance with some preferred embodiments of the present invention, cell piercing device (CPD) 20, shown in FIGS. 3 and 4, comprises container 22, respectively matching cap 24, cell interacting surface (hereinafter CIS) pedestal 26 and CIS 28. Container 22 is essentially cylindrical and comprises an anterior opening allowing loading and discharging of solutions. Container 22 may include one or more trunnions (not shown), adapted for accommodation of CPD 20 in standard centrifuges. Respectively matching cap 24 is preferably somewhat elongated to encompass a volume substantially similar to container 22. Cap 24 is capable of hermetically sealing container 22, thereby preventing spillage of the liquid content thereof and penetration of contaminants and microorganisms thereto. Screw threading 23 is employed at the interface between container 22 and cap 24, for securing the latter to the former.

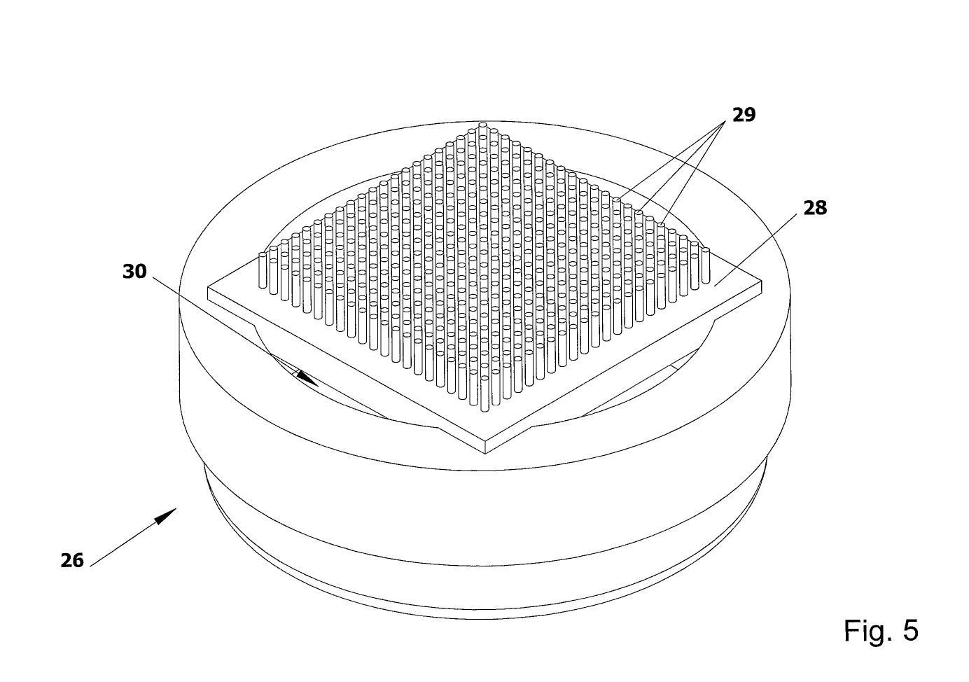

CIS pedestal 26 is disposed within container 22 spaced from the bottom; this achieved by the interior cylindrical stepped shoulder at the bottom of container 22. To depict the mounting of CIS onto pedestal 26, reference is now made also to FIG. 5. CIS 28, having an essentially rectangular form disposed in respective recesses within the annular structure of pedestal 26 and affixed therein, while extensions or prongs 29 facing an upward direction, away from pedestal 26. Slits 30 are preferably formed at the margins of CIS 28, allowing solutions to infiltrate theretrough, into the compartment formed below CIS 28.

CIS 28 comprises a plurality of micro- and/or nano-scale extensions or prongs 29. Extensions or prongs 29 preferably have the diameter and length of ˜90 nm and ˜6 μm, respectively. CIS 28 is preferably produced as disclosed by P. Yang, in ‘Interfacing Silicon Nanowires with Mammalian Cells’ as published in Journal of the American Chemical Society, volume 129, number 23, on 22 May 2007. The molecule subject to the delivery application is preferably nucleic acids and particularly DNA. The linking mechanisms to be employed are preferably the ones disclosed by T. E. McKnight, in ‘Intracellular integration of synthetic nanostructures with viable cells for controlled biochemical manipulation’, as published by Institute of Physics Publishing in Nanotechnology, issue 14 pages 551 to 556, on 9 Apr. 2003 and in US 2004/0197909 or by C. R. Bertozzi, in ‘A cell nanoinjector based on carbon nanotubes’, as by published in PNAS, volume 104, number 20, on 15 May 2007.

The CPD is typically filled with a solution up to a substantial portion of its volume, whereas the respectively remaining portion of its volume is occupied by ambient air or any other gaseous substance; thereby providing for a gaseous bubble inside the CPD. In this respect it is noted that the volume of the lower compartment formed between CIS pedestal 26 and the bottom of container 22 is preferably somewhat larger than the aforementioned gaseous bubble inside the CPD; thereby providing for a continuous sustention of the sells in an aqueous solution environment.

CPD 20 is loaded with solution containing the target cells, while container 22 is held in an essentially upward orientation. The step of loading can be preceded with a pre-treatment step of incubating CIS 28 with the target molecules to be delivered in order to link attach, or absorb the same thereto; essentially as described in the aforementioned references.

Cap 24 is then tightened on container 22 to seal it. CPD 20 is placed in a centrifuge, while cap 24 is oriented essentially towards the rotational axis and subsequently centrifuged. Upon the completion of the interaction, CPD 20 is placed in the centrifuge essentially reciprocally oriented, while container 22 faces the rotational axis and subsequently centrifuged.

Cap 24 is then removed from container 22, while cap 24 is held in essentially upward orientation and the anterior opening of container 22 faces downwards. The cells can thence be collected from cap 24.

It will be appreciated by persons skilled in the art that the present invention is not limited by what has been particularly shown and described herein above. Thus the CPD of the invention can be implemented with a plurality of CISs disposed in different places and oriented in various directions. Rather the scope of the invention is defined by the claims which follow:

1. A cell piercing device comprising:
a container comprising an anterior opening allowing loading and discharging of solutions and a bottom;
a respectively matching cap, capable of sealing said container;
at least one cell interacting surface, disposed in said container, said cell interacting surface comprising a plurality of extensions or prongs, and
a pedestal, spaced apart from said bottom of said container.

2. (canceled)

3. The device as in claim 1, further comprising at least one trunnion.

4. The device as in claim 1, adapted to be accommodated in a standard centrifuge.

5. The device as in claim 1, wherein said respectively matching cap is somewhat elongated to encompass a volume substantially similar to that of said container.

6. The device as in claim 1, wherein said respectively matching cap is capable of hermetically sealing said container, thereby preventing spillage of the liquid content thereof and penetration of contaminants and microorganisms thereto.

7. The device as in claim 1, wherein a screw threading is employed at the interface between said container and said cap, for securing the cap to the container.

8. The device as in claim 1, wherein said cell interacting surface is somewhat spaced from the bottom of said container thereby forming a lower compartment below said cell interacting surface.

9. The device as in claim 1, wherein slits are formed at the margins of said cell interacting surface, allowing solutions to infiltrate therethrough.

10. A method of facilitating an interaction with the interior of cells, said method comprising the steps of:
loading a cell piercing device into a container, through an anterior opening thereof, a liquid solution containing said cells;
tightening a respectively matching cap, thereby sealing said container;
centrifuging said device while extensions or prongs protruding from a cell 1 interacting surface, disposed in said container, are oriented essentially within the direction of the vector of the centripetal force; whereby said cells are urged by 1 the centrifugal force towards said cell interacting surface and consequently pierced by said extensions or prongs and spitted onto them;
centrifuging said device while said extensions or prongs are oriented essentially within the direction of the vector of the centrifugal force; whereby said cells are urged by the centrifugal force away from said cell interacting surface and consequently driven off said extensions or prongs and released into the interior of said container;
removing said cap from said container and collecting said cells.

11. The method as in claim 10, wherein said steps of centrifuging said device while extensions or prongs protruding from a cell interacting surface oriented essentially within the direction of the vector of the centripetal force, and centrifuging said device while said extensions or prongs are oriented essentially within the direction of the vector of the centrifugal force are performed by:
placing said device in a centrifuge, while said cap is oriented essentially towards the rotational axis;
centrifuging said device;
placing said device in a centrifuge essentially reciprocally oriented, while container faces the rotational axis; and
centrifuging said device.

12. (canceled)

13. The method as in claim 10, wherein said step of loading said container of cell piercing device, through an anterior opening thereof, with a liquid solution containing said cells comprises filling a substantial portion of said container with said solution, whereas a remaining portion of said container is occupied by a gaseous substance.

14. The method as in claim 13, wherein a compartment is formed between said cell interacting surface and the bottom of said container and wherein the volume of said compartment is somewhat larger than said volume occupied by a gaseous substance; thereby providing for a continuous suspension of the cells in an aqueous solution environment.
